(19) United States
(12) Patent Application Publication
(10) Pub. No.: US 2026/0315265 A1
(43) Pub. Date: Oct. 15, 2026

(54) PAD HAVING MULTIPLE INTERCONNECTED, MOVABLE RAISED PORTIONS

(71) Applicant: BAUERFEIND AG, Zeulenroda-Triebes (DE)

(72) Inventors: André BECK, Zeulenroda-Triebes (DE); Robin WÖLLNER, Zeulenroda-Triebes (DE); Beatrix BAUERFEIND-JOHNSON, Zeulenroda-Triebes (DE)

(21) Appl. No.: 19/484,983

(22) PCT Filed: May 29, 2024
(86) PCT No.: PCT/EP2024/064739

§ 371 Date: Nov. 17, 2025

(30) Foreign Application Priority Data
Jun. 2, 2023 (DE) 10 2023 205 185.0

(51) Int. Cl. A61F 5/30

(52) U.S. Cl. CPC A61F 5/30

(57) ABSTRACT

Pads, particularly for orthopedic aids, include at least two nub elements and at least one counter-bearing element. The pads may be used in orthopedic aids or sports aid devices, in particular orthoses or bandages, as well as orthopedic aids or sports aid devices, in particular orthoses or bandages, which comprise pads according to the invention.

### CROSS-REFERENCE TO RELATED APPLICATIONS

This application is a U.S. National Phase Application under 35 U.S.C. 371 of International Application No. PCT/EP2024/064739, filed on May 29, 2024, which claims the benefit of German Patent Application No. 10 2023 205 185.0, filed on Jun. 2, 2023. The entire disclosures of the above applications are incorporated herein by reference.

### FIELD

The invention relates to pads, in particular for orthopedic aids, comprising at least two nub elements and at least one counter-bearing element. The invention also relates to the use of the pads according to the invention in orthopedic aids or sports aid devices, in particular orthoses or bandages, as well as orthopedic aids or sports aid devices, in particular orthoses or bandages, which comprise pads according to the invention.

### BACKGROUND

This section provides background information related to the present disclosure which is not necessarily prior art.

Pads are known in various embodiments and for various prophylactic and therapeutic applications. Various pads are known, for example, from DE 27 22 563 C2, EP 0 598 291 A1, and EP 0 600 218 A2.

Pads made of two materials or components are also known from DE 297 01 001 U1. EP 0 496 071 A1 describes a pressure pad made of a softer material in which at least one friction core made of a hard or incompressible material is arranged. This friction core is intended to provide friction massage, i.e., massage through movement friction of pain points, which is generated by the movement of the core relative to the soft tissue of the pad carrier.

DE 10 2019 212 740 B3 describes raised portions that protrude from the pad toward the wearer, wherein at least two viscoelastic movable cone-shaped raised portions are interconnected.

The pads from the prior art can exert pressure on the adjacent tissue over a large area or distribute and/or shield the pressure exerted on the surface, but with these pads it is not possible, or only possible to an insufficient degree, to exert specific pressure, for example stronger or weaker pressure, on specific points.

Furthermore, with prior art pads, pressure is applied vertically to the skin. Lateral pressure, which particularly causes the pressure elements to move and thus produces a massage effect beyond simple pressure, is therefore not possible.

Furthermore, the pressure exerted by the prior art pressure bodies is often insufficiently strong.

### SUMMARY

This section provides a general summary of the disclosure, and is not a comprehensive disclosure of its full scope or all of its features.

The technical problem underlying the present invention is therefore the provision of improved pads, in particular pads that can exert specific, precise pressure in addition to flat pressure. In particular, the technical problem underlying the present invention is the provision of a pad that leads to an improved massage effect, in particular a massage effect through different movements of the pressure body. In particular, the technical problem underlying the present invention is the provision of a pad which, when pressure is applied to the pad base element in a varying manner, causes its nub-like raised portions to tilt away in a defined manner and thus follow the primarily desired massage direction. In the prior art, no adequate solution has yet been found to the problem that nub-like raised portions on pads, similar to the fingers of a massage therapist/physical therapist, perform repetitive movements in a defined direction during manual therapy.

The technical problem underlying the invention is solved by the provision of a pad for orthopedic aids or sports aid devices, comprising a pad base element made of a first material, wherein the pad base element comprises an active surface and a counter-bearing surface, wherein at least one nub element is assigned to the pad base element, wherein the nub element comprises at least two nub areas that protrude from the active surface of the pad base element, wherein the nub element comprises at least one counter-bearing area that protrudes from the counter-bearing surface of the pad base element, wherein the at least one counter-bearing area is interconnected with the at least two nub areas.

In the present invention, the nub element thus protrudes not only from the active surface of the pad base element, as is the case with conventional pads, but also from the counter-bearing surface. In cross-section, this results in three planes of the pad: In the middle is the pad base plane, in which the pad base element is located; from this pad base plane in the direction of the person wearing the pad is the active plane, in which the nub areas of the nub element are located; and in the other direction of the pad base plane, in particular, if preferably present, in the direction of the pad carrier, is the counter-bearing plane, in which the counter-bearing area of the nub element is located. Between the active plane and the counter-bearing plane is the pad base plane, which can be regarded as the “thickness” of the pad base body.

This allows the at least one nub element to be advantageously controlled by means of the counter-bearing area in a second plane facing away from the user's body, i.e., the counter-bearing plane. This “external” control advantageously increases the pressure exerted by the nub elements. The external pressure, for example from the pad carrier, for example in the form of a knitted fabric or a bandage base body, on these counter-bearing areas of the nub elements in the counter-bearing plane controls the nub elements and also generates a different massaging movement for the mobile user through the nub areas on the active surface facing the body, i.e., in the active plane.

If multiple nub areas in the counter-bearing plane are interconnected, they advantageously perform a spreading movement when force is applied from the counter-bearing plane. If, on the other hand, the force is applied inversely from the active plane to the counter-bearing plane, the nub areas perform a gripping movement, i.e., they practically fall toward an imaginary common center. This allows for different massage effects. These effects cannot be reproduced by a nub attached to a spoke without a counter-bearing element, as described in DE 10 2019 212 740 B3.

For the function according to the invention, the counterpressure does not necessarily have to be generated by the pad carrier. It is also possible for this counterpressure to be generated, for example, by a chair back, a hand placed on the pad, etc., without the pad carrier covering the relevant area of the pad. The nub-spring technology according to the invention via the counter-bearing area can also take effect if the pad is applied directly to the body part, for example, glued, as long as an additional force can be applied in the counter-bearing plane. Thus, a pad carrier can be advantageous, but is only optional and not mandatory.

The combination of pressure on both sides of the pad creates, in an advantageous manner, a complex movement pattern of the nub elements, which varies with each movement of the patient.

It turned out to be advantageous that this lateral movement of the nub elements also proceeds chaotically depending on the direction of the applied force vector.

The elements interconnected according to the invention, i.e., especially preferred, the nub elements, spring much more intensively than raised portions from the prior art, but also in a more controlled manner than without guided movement. A specifically controlled tilting or springing movement is never completely identical to the movement that follows it, as slightly different movement patterns arise repeatedly depending on the pressure or tension on the counter-bearing. This effect is advantageous because the mechanoreceptors located directly under the skin surface find it more difficult to adjust to the constantly changing spring movement of the nub elements. This leads to a prolonged massage effect. One could also speak of a targeted-chaotic movement that significantly reduces unwanted movements of the nub elements.

Since manual therapy involves “chaotic” massage, as no two grips by the masseur are 100% identical, the massage effect produced by the pad according to the invention is more similar to that of a manual massage than the massage effect produced by conventional pads. The combination of nub elements with counter-bearing advantageously results in movable raised portions that enable an intermittent massage principle. While immovable raised portions on a pad only have a pressure effect on the skin sensors, which, however, become accustomed to the pressure after a relatively short time and no longer send out effective signals, the movable nub elements according to the invention lead to alternating pressure, such as that exerted by a therapist during a massage, for example, so that the pressure effect on the lymph and blood vessels consists. This can be particularly advantageous, for example, in a knee pad, where a corresponding massage effect is applied to the meniscus base and Hoffa's fat pad. The nub elements move in an advantageous targeted-chaotic manner, and in different directions depending on the force application vectors.

In the context of the present invention, a pad is understood to be, in particular, a pressure pad whose shape is formed in particular by the pad base element. The pad base element can comprise any suitable shape. Conventional pads typically comprise flat base surfaces as the pad base element. Protrusions, in this case particularly the nub elements, can then protrude from such a base surface. A pad according to the invention comprises, in particular, a first base surface or base area which faces the user when the pad is used. This is the active surface. Preferably, a pad is designed to be essentially flat. Such a pad then comprises a second base surface or base area which faces away from the user when the pad is used. This is the counter-bearing surface.

The typical use of a pad is known to a person skilled in the art. Pads, including those according to the invention, are used particularly to exert pressure and, in some cases, a complementary massage effect on certain parts of the body, for example in the back or knee area. The pads are positioned and pressed onto the corresponding area of the body using additional devices, particularly sports aid devices, bandages, or orthoses.

Preferably, the pad base element is plate-shaped, i.e., a plate. The plate can be flat or curved or arched. The plate can comprise one or multiple recesses.

Nub areas and counter-bearing area can be positioned next to each other or on top of each other on the nub element. For example, the nub areas and counter-bearing area can be two different bodies that protrude in different directions, or they can be formed from a single body that protrudes from both the active surface and the counter-bearing surface.

In a preferred embodiment, the nub element comprises a bar element. The bar element can be located between the counter-bearing area and the pad base body, or the counter-bearing area forms the bar element.

Preferably, the at least one counter-bearing area is a bar element.

The bar element advantageously causes a defined, controlled spring deflection movement to be generated, which can be amplified by an optional protrusion, which causes a relative lateral movement on the cones sitting on the optional protrusion, for example, and not a “floating spring” movement that is possible in all directions.

In a preferred embodiment, the at least two nub areas are movable.

Preferably, the nub areas are pressure-enhancing elements. A pressure-enhancing element serves to exert greater pressure at specific points than the surface pressure exerted by the pad base element.

Preferably, the at least two nub areas are cone-shaped raised portions. Suitable shapes are known to the person skilled in the art.

In a preferred embodiment, the pad comprises at least two nub elements. In a preferred embodiment, the pad comprises at least two and at most twenty nub elements. In a preferred embodiment, the pad comprises a plurality of nub elements.

The plurality of nub elements can be positioned on the pad base element so that when the pad is used, they exert precise pressure on specific points, for example on trigger points, acupuncture points, muscles, tendons, muscle or tendon attachments, or infrapatellar fat bodies.

In addition to the at least one nub element, the pad may also comprise additional friction elements or other pad pressure elements, for example made from the pad base element material.

In a preferred embodiment, the at least one nub element consists of a softer material than the pad base element. In a preferred embodiment, the at least one nub element is made of a softer material than the pad base element.

In one embodiment, the pad base element and the at least one nub element are made in one piece and/or from the same material. In one embodiment, the pad base element and the at least one nub element are made in one piece. In one embodiment, the pad base element and the at least one nub element are made from the same material.

In a preferred embodiment, the pad base element comprises at least one hole, wherein the at least one nub element is positioned in the hole.

In a preferred embodiment, the nub areas can be connected with the pad base element, meaning that the nub areas are located between the pad base element and the counter-bearing area. In such an embodiment, it is preferred that multiple nub elements be interconnected with one counter-bearing area or with multiple counter-bearing areas.

In a preferred embodiment, the at least two nub areas are positioned at the edge of the hole, and the counter-bearing area is positioned in the hole.

In a preferred embodiment, three, four, or five nub areas are positioned at the edge of the hole, and the counter-bearing area is positioned in the hole.

The effect of the nub elements in combination with the counter-bearing element can be advantageously enhanced by further raising the seat of the nub elements at the edge of the recess or hole, for example, if there is a protrusion or bead at the edge of the hole or recess on which the nub elements sit, as this extends the fall or spread distance, which intensifies the massage effect.

In a preferred embodiment, the nub element comprises a protrusion, particularly a protrusion at the edge of the hole. In a preferred embodiment, the at least two nub areas are positioned on the protrusion.

Preferably, the at least one nub element comprises two, three, four, five, six, seven, eight, nine, ten, or eleven nub areas.

Preferably, the at least one nub element comprises two, three, or four nub areas.

Preferably, the at least one nub element comprises at least three nub areas.

Preferably, the at least one nub element comprises three, four, or five nub areas.

Preferably, the at least one nub element comprises three or four nub areas.

Preferably, the at least one nub element comprises three nub areas. Preferably, the at least one nub element comprises four nub areas.

When not pressed down, the nub area preferably protrudes from the pad base body, i.e., the pad base plane, at an angle of approximately 90°, particularly 90°, toward the user. However, when not pressed down, the nub area may also protrude from the pad base body, i.e., the pad base plane, at a different angle toward the user.

For greater flexibility of the at least one nub element, it may comprise thinnings or thickenings, for example in the nub area, in the counter-bearing area between the nub area and the counter-bearing area, or between the counter-bearing area and the end of the nub element interconnected with the pad base element. Film hinges, wedge shapes, and the like are also possible.

The pad comprises at least one nub element, preferably two, three, four, five, ten, or more nub elements.

It is particularly provided that multiple, particularly two, three, four, or five nub areas are interconnected with each other via counter-bearing areas, i.e., in the counter-bearing plane, particularly via bars. This allows the formation of 2-piece, 3-piece, 4-piece, or other “nub nests” from multiple interconnected nub areas.

In a preferred embodiment, the pad base element is at least partially embedded in a sheathing. In a preferred embodiment, the pad base element is completely embedded in a sheathing.

This means that the shape of the sheathing can determine the shape of the pad.

Preferably, the pad base element is therefore completely enclosed by the sheathing, for example, cast into the sheathing.

The preferred complete sheathing of the at least one pad base element with a sheathing material, particularly a softer sheathing material, for example silicone, not only results in the described application-related advantage, but also, in an advantageous manner, in a simpler manufacturing option for the pad according to the invention, since the second base surface of the pad base body, which faces away from the user, i.e., the counter-layer surface, can be easily manufactured flat despite the counter-layer area of the nub element, but it can also comprise protrusions and/or indentations.

The protrusion at the edge of the hole near a nub element can be made of a third material that can be softer or harder than the material from which the pad base element consists.

Suitable materials for a sheathing and the protrusion are known to the person skilled in the art and may in particular be materials used for conventional flexible pads consisting of one component.

Preferably, the sheathing or protrusion comprises a viscoelastic material at least in the area of the at least two nubs. Preferably, the sheathing is viscoelastic at least in a partial area. Preferably, the sheathing is viscoelastic. Preferably, the sheathing consists of a viscoelastic material.

Preferred materials for the sheathing or protrusion are plastic, silicone, or rubber. The material may also be thermoplastic elastomers. Polyurethanes, for example, are also suitable.

Preferably, the material of the sheathing or protrusion is flexible and/or stretchable so that the sheathing can adapt to the user's body shape when the pad is applied.

Preferably, the sheathing or protrusion material is not a textile material. However, in an alternative embodiment, the sheathing or protrusion may also be additionally surrounded by a textile sheathing or otherwise coated.

The sheathing material and/or protrusion may, of course, also consist of mixtures of materials, particularly of at least two of the materials mentioned.

Preferably, the material of the pad base element is a flexible material, particularly a flexible plastic. In a preferred embodiment, the material of the pad base element is plastic, silicone, or rubber. The material of the pad base element may, of course, also consist of mixtures of materials, particularly of at least two of the materials mentioned. Preferably, the pad base element and the at least one nub element consist of the same material or are made of the same material, i.e., they are one-piece. Preferably, the material of the nub element is a flexible material, particularly a flexible plastic. In a preferred embodiment, the material of the nub element is plastic, silicone, or rubber. The material of the nub element can, of course, also consist of mixtures of materials, particularly of at least two of the materials mentioned.

Thus, the nub element pad base element construction according to the invention can be advantageously and easily manufactured, for example, by means of injection molding technology.

However, the nub elements, particularly their nub areas, can also be manufactured from multiple components.

According to the invention, the second material of the sheathing is softer than the first material of the pad base element.

In a preferred embodiment, the material of the sheathing has a Shore hardness of at least 10 Shore OO and at most 50 Shore OO.

Preferably, the material of the sheathing has a hardness of at most 45 Shore OO, preferably at most 40 Shore OO, and especially preferably at most 30 Shore OO. Preferably, the material of the sheathing has a hardness of at most 25 Shore OO, preferably at most 20 Shore OO, and especially preferably at most 19 Shore OO. Preferably, the material of the sheathing has a hardness of at least 10 Shore OO, particularly at least 14 Shore OO, and especially preferably at least 15 Shore OO.

In a preferred embodiment, the material of the pad base element has a Shore hardness of at least 10 Shore D, preferably at least 14 Shore D and at most 80 Shore d. In a preferred embodiment, the material of the pad base element has a Shore hardness of at least 20 Shore D and at most 80 Shore D.

In a preferred embodiment, the material of the at least one pad base element comprises a hardness of at least 20 Shore D and at most 60 Shore D.

In a preferred embodiment, the material of the at least one pad base element comprises a hardness of at least 20 Shore D, more preferably of at least 25 Shore D.

In a preferred embodiment, the material of the at least one pad base element comprises a hardness of at most 50 Shore D. In a preferred embodiment, the material of the at least one pad base element comprises a hardness of at most 49 Shore D, more preferably at most 45 Shore D.

Preferably, the material of the at least one pad base element comprises a hardness of at least 35 to at most 45 Shore D. Preferably, the material of the at least one pad base element comprises a hardness of about 40 Shore D.

In a preferred embodiment, the material of the nub element has a Shore hardness of at least 10 Shore A, preferably at least 14 Shore A and at most 80 Shore A. In a preferred embodiment, the material of the nub element has a Shore hardness of at least 20 Shore A and at most 60 Shore A.

In a preferred embodiment, the material of the at least one nub element comprises a hardness of at least 25 Shore A and at most 50 Shore A.

In a preferred embodiment, the material of the at least one nub element comprises a hardness of at least 30 Shore A, more preferably at least 35 Shore A.

Preferably, the material of the at least one nub element comprises the same hardness as the protrusion and the pad base element. In a preferred embodiment, the material of the pad base element has a Shore hardness of at least 10 Shore A, preferably at least 14 Shore A and at most 80 Shore A. In a preferred embodiment, the material of the pad base element has a Shore hardness of at least 20 Shore A and at most 80 Shore A.

In a preferred embodiment, the material of the at least one pad base element and that of the at least one protrusion comprise a hardness of at least 20 Shore A and at most 60 Shore A.

In a preferred embodiment, the material of the at least one pad base element and that of the at least one protrusion comprise a hardness of at least 20 Shore A, more preferably of at least 25 Shore A.

In a preferred embodiment, the material of the at least one pad base element and that of the at least one protrusion comprise a hardness of at most 50 Shore A. In a preferred embodiment, the material of the at least one pad base element and that of the at least one protrusion comprise a hardness of at most 49 Shore A, more preferred of at most 45 Shore A.

Preferably, the material of the at least one pad base element and that of the at least one protrusion comprise a hardness of at least 35 to at most 45 Shore A. Preferably, the material of the at least one pad base element comprises a hardness of about 40 Shore A.

In a preferred embodiment, the material of the sheathing has a Shore hardness of at least 10 Shore OO and at most 50 Shore OO and/or the material of the pad base element has a Shore hardness of at least 10 Shore D, preferably at least 14 Shore D and at most 80 Shore D.

In a preferred embodiment, the material of the pad base element has a Shore hardness of at least 10 Shore-D and at most 80 Shore-D material, wherein the sheathing preferably has a Shore hardness of at least 10 Shore-OO and at most 50 Shore-OO. In a preferred embodiment, the material of the sheathing has a Shore hardness of at least 10 Shore-OO and at most 50 Shore-OO.

In a preferred embodiment, the pad comprises a sheathing, wherein the nub element is not firmly connected to the pad base element and is preferably embedded in the sheathing. The nub element is thus mounted and positioned in the sheathing, particularly floatingly mounted, in such a way that the nub area protrudes from the active surface of the pad base element and the counter-bearing area protrudes from the counter-bearing surface of the pad base element without the nub element being firmly interconnected with the pad base element by a bar or in any other way. The nub element is therefore positioned relative to the pad base element solely by means of the sheathing. Preferably, the pad base element comprises at least one hole, wherein the at least one nub element is positioned in the hole and thus protruding through it. Surprisingly, it was found that a floatingly mounted nub element also experiences an increase in effectiveness in the active plane due to counterpressure. The pad, particularly the pad base element, can have any shape, particularly any pad shape known to the person skilled in the art.

For example, the pad may be ring-shaped, particularly when used as a patella pad. Alternatively, the shape may also be a flat continuous surface. Wing-shaped attachments may be provided on one or multiple sides of the pads.

In a preferred embodiment, the pad is a knee pad, an ankle pad, a back pad, an elbow pad, a pad for the arm area, or a pad for the abdominal area. In a preferred embodiment, the pad is a knee pad or a back pad. In a preferred embodiment, the pad is a knee pad. In a preferred embodiment, the pad is a back pad.

A pad is preferred, wherein the at least one nub element of the pad is positioned in the pad base element such that, when the pad is applied, it is located in the area of a trigger point and can exert pressure on the trigger point.

A pad according to the invention for use in trigger point therapy is preferred, wherein the at least one nub element of the pad is preferably positioned in the pad base element such that it presses on a trigger point when the pad is worn.

The aim of trigger point therapy is to eliminate so-called myofascial trigger points. These are locally limited hardenings in the skeletal muscles.

A pad, particularly a knee pad, as shown in the figures, is preferred.

The pad according to the invention is preferably not a shoe insole or foot insole.

The pad is preferred for an orthopedic aid or a sports aid device. The pad is preferred part of an orthopedic aid or a sports aid device. The orthopedic aid or sports aid device can be an orthosis or a bandage.

The present invention also relates to a sports aid device or an orthopedic aid, particularly an orthosis or a bandage, comprising a pad according to the invention. The present invention also relates to a sports aid device comprising at least one pad according to the invention. The present invention also relates to an orthopedic aid comprising at least one pad according to the invention. The present invention also relates to an orthosis or a bandage comprising a pad according to the invention.

Preferred is a sports aid device, orthosis, or bandage comprising at least one pad according to the invention and a pad carrier, in particular a knitted or crocheted fabric, wherein the pad carrier rests against the counter-bearing surface of the pad base element of the pad.

Preferably, the pad carrier is a knitted or crocheted fabric.

The pad can, for example, be attached to the pad carrier in a pocket. Alternatively, it can also be attached directly to the pad carrier, for example, via Velcro fasteners or buttons.

Preferably, the pad can be attached to the pad carrier reversibly and variably in position.

Preferably, the orthosis is an elastic knitted orthosis or the bandage is an elastic knitted bandage, particularly a knee bandage.

The present invention also relates to the use of a pad according to the invention as a pressure pad, preferably in trigger point therapy.

The present invention also relates to a method for trigger point therapy, in which a pad according to the invention is applied to a user in a first step in such a way that the at least one nub element presses on a trigger point, and in a second step, the pad according to the invention is worn by the user in this position for a certain period of time.

Further preferred embodiments of the invention are set out in the subclaims and subsidiary claims.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

The invention is described in more detail with reference to the figures below, without the embodiments of the invention shown therein being understood as limiting.

FIG. 1 shows a schematic cross-section of a section of a pad base element with a nub element;

FIG. 2A shows the active surface of the pad base element of a back pad according to the invention, the pad base element facing the wearer;

FIG. 2B shows the counter-bearing surface of the pad base element of the back pad according to the invention, the pad base element facing away from the wearer and toward an optional pad carrier;

FIGS. 3A and 3B show two further views of the back pad according to the invention from FIGS. 2A and 2B;

FIG. 4 shows a cross-section of a section of a pad base element with a nub element;

FIG. 5A shows a top view of the side of the pad and the pad base element of the knee pad according to the invention facing away from the body;

FIG. 5B shows a top view of the side of the pad and the pad base element of the knee pad according to the invention facing the body;

FIG. 6A shows a top view of the side of the pad base element facing away from the body; and

FIG. 6B shows a top view of the side of the pad base element facing the body.

### DETAILED DESCRIPTION

Example embodiments will now be described more fully with reference to the accompanying drawings.

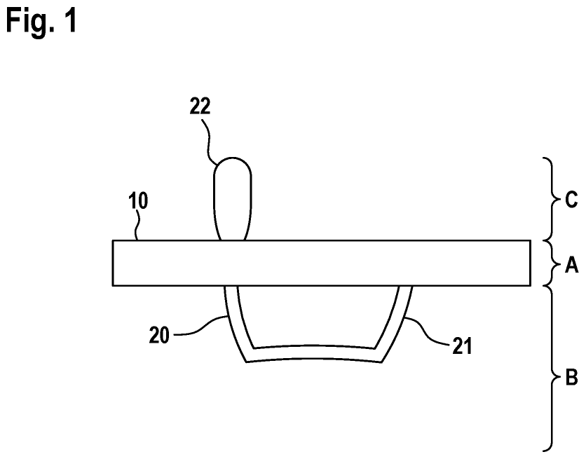

FIG. 1 shows a schematic cross-section of a section of a pad base element (10) with a nub element (20). The pad base element (10) extends over a large area and, in the abstract structure of the pad in three planes, lies in the middle plane, i.e., the pad base plane (A). The nub element (20) is interconnected with the pad base element (10) via the counter-bearing area (21). The counter-bearing area (21) lies below the pad base plane (A) and thus in the counter-bearing plane (B). The counter-bearing area (21) is connected to one of at least two nub areas (22) with which the nub element (20) ends. The nub area (22) is located above the pad base plane (A) and thus in the active plane (C). The pad base plane (A) is therefore located in the middle, with the active plane (C) located between this pad base plane (A) and the person wearing the pad, and the counter-bearing plane (B) located in the other direction of the pad base plane (A), particularly, if preferably present, in the direction of the pad carrier. The pad base plane (A) is therefore located between the active plane (C) and the counter-bearing plane (B), which be regarded as the “thickness” of the pad base body (10).

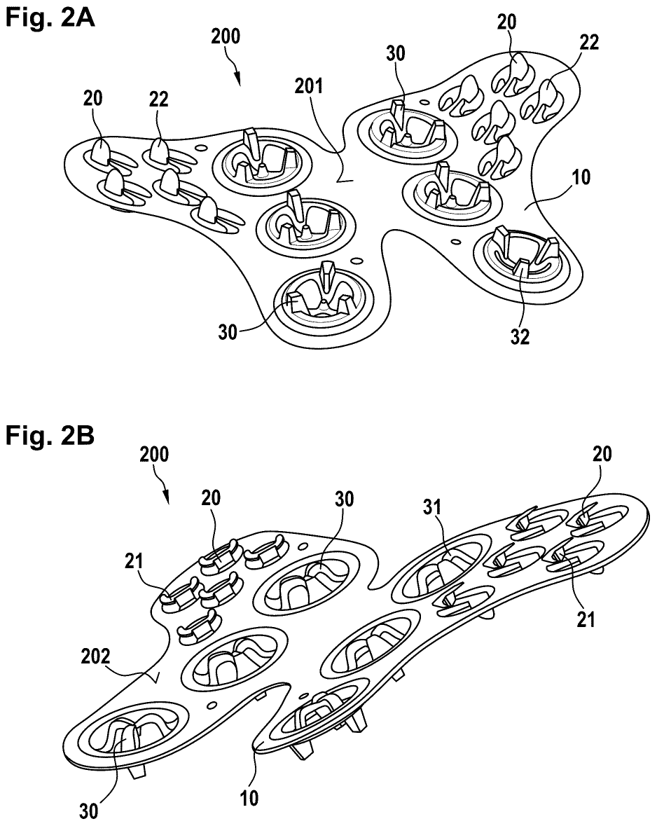

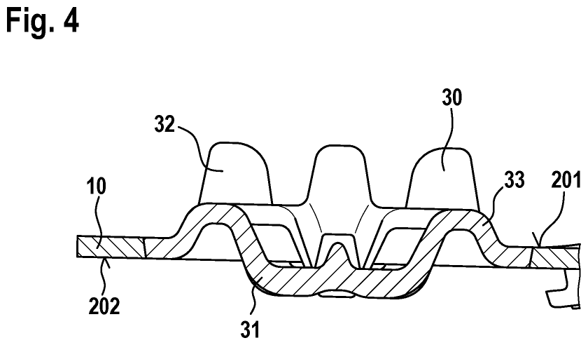

FIGS. 2A and 2B show a back pad (200) according to the invention. FIG. 2A shows the active surface (201) of the pad base element (10) facing the wearer. FIG. 2B shows the counter-bearing surface (202) of the pad base element (10) facing away from the wearer and toward an optional pad carrier. The back pad comprises a flat pad base element (10) with multiple nub elements (20,30) distributed across the pad base element (10). Ten nub elements are nub elements (20) with only one nub area, while six further nub elements (30) are nub elements (30) according to the invention, each comprising three interconnected nub areas (32). The exact structure of the nub elements (20,30) can be seen in FIG. 4.

All nub elements (20,30) have in common that they protrude with their nub areas (22,32) from the active surface (201) of the pad base element (10) and protrude with their counter-bearing area (21,31) from the counter-bearing surface (202) of the pad base element (10).

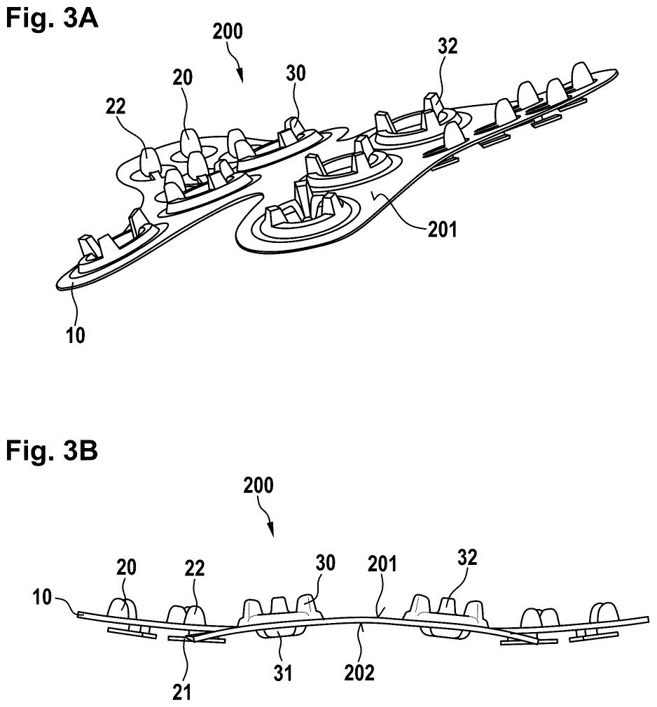

FIGS. 3A and 3B show the back pad (200) according to the invention from FIGS. 2A and 2B from two other perspectives. FIG. 3A shows an oblique view of the active surface (201) of the pad base element (10) facing the wearer. FIG. 3B shows the side view. It can again be clearly seen how the nub elements (20,30) protrude from the active surface (201) of the pad base element (10) with their nub area (22,32) and protrude from the counter-bearing surface (202) of the pad base element (10) with their counter-bearing area (21,31).

FIG. 4 shows a cross-section of the pad base element (10) of the pad shown in FIGS. 2A, 2B, 3A, and 3B. A nub element (30) with three interconnected nub areas (32) positioned on a protrusion (33) can be seen. The nub element (30) is interconnected with the pad base element (10) via the nub areas (32). The nub area (32) is located above the active surface (201). The counter-bearing area (31) adjoins the nub area (32). The counter-bearing area (31) is located below the counter-bearing surface (202). The three nub areas (32) are interconnected via the counter-bearing area (31) and thus form a “nub nest”. The nub element (30) can consist of a softer material than the pad base element (10).

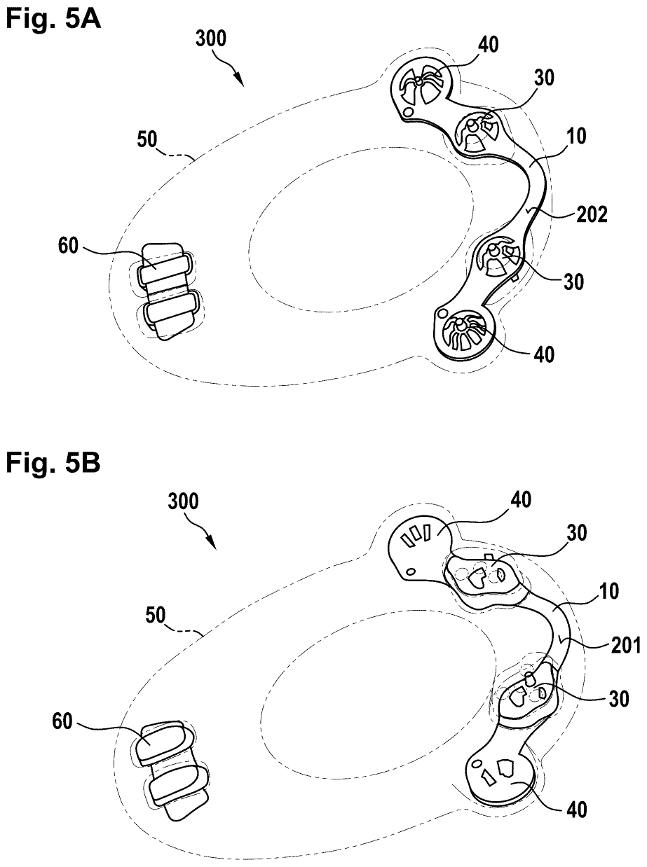

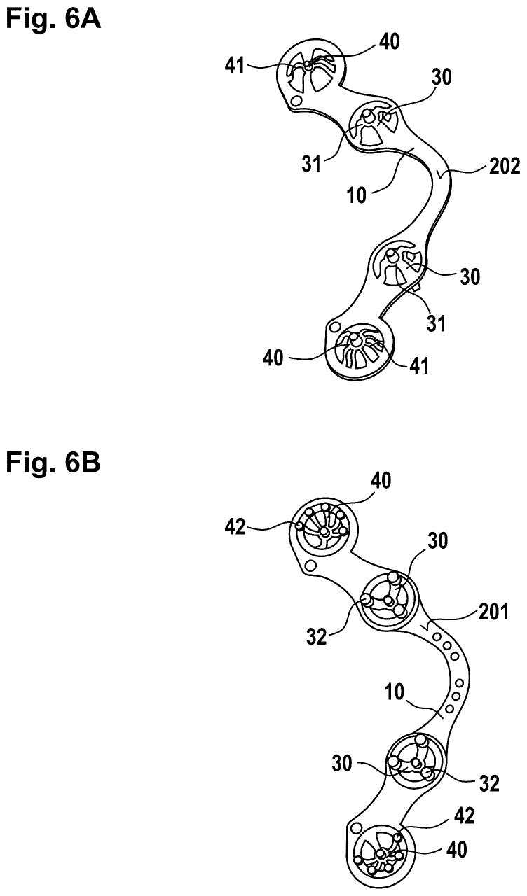

FIGS. 5A and 5B show a knee pad (300) according to the invention. This comprises a pad base element (10) with two nub elements (30) with three nub areas and two nub elements (40) with five nub areas, a softer sheathing (50) in the typical shape of a knee pad, in which the pad base element (10) is molded, and a further, optional pressure element (60) also molded into this. FIG. 5A shows a top view of the side of the pad (300) and the pad base element (10) facing away from the body, i.e., the counter-bearing surface (202). FIG. 5B shows a top view of the side of the pad (300) and the pad base element (10) facing the body, i.e., the active surface (201). The exact structure of the pad base element (10) and the nub elements (30,40) can be seen in FIGS. 6A and 6B.

FIGS. 6A and 6B show the pad base element (10) of the knee pad from FIGS. 5A and 5B. FIG. 6A shows a top view of the side of the pad base element (10) facing away from the body, i.e., the counter-bearing surface (202). FIG. 6B shows a top view of the side of the pad base element (10) facing the body, i.e., the active surface (201). The pad base element (10) has two nub elements (30), each with three nub areas (32), and two nub elements (40), each with five nub areas (42). The nub elements (30,40) are each positioned in a hole in the pad base element (10). The nub area (32,42) is located above the active surface (201). The nub area (32,42) each adjoins the counter-bearing area (31,41). The counter-bearing area (31,41) is located below the counter-bearing surface (202). The respective nub areas (32,42) are interconnected via the respective counter-bearing area (31,41) and thus form a “nub nest.”

1. A pad for orthopedic aids or sports aid devices, comprising a pad base element made of a first material, wherein the pad base element comprises an active surface and a counter-bearing surface, wherein at least one nub element is assigned to the pad base element, wherein the nub element comprises at least two nub areas which protrude from the active surface of the pad base element, wherein the nub element comprises at least one counter-bearing area protruding from the counter-bearing surface of the pad base element, wherein the at least one counter-bearing area is connected to the at least two nub areas, and wherein the pad is a knee pad, an ankle pad, a back pad, an elbow pad, a pad for an arm area, or a pad for an abdominal area.

2. The pad according to claim 1, wherein the at least one counter-bearing area is a bar element.

3. The pad according to claim 1, wherein the at least two nub areas are cone-shaped raised portions.

4. The pad according to claim 1, wherein the pad base element comprises at least one hole, wherein the at least one nub element is positioned in the hole.

5. The pad according to claim 4, wherein the at least two nub areas are positioned at an edge of the hole and the counter-bearing area is positioned in the hole.

6. The pad according to claim 1, wherein the at least one nub element comprises three or four nub areas.

7. The pad according to claim 1, wherein the at least one nub element consists of a softer material than the pad base element.

8. The pad according to claim 1, wherein the at least one nub element comprises a protrusion at the edge of a hole located in its area.

9. The pad according to claim 1, wherein the pad base element is at least partially embedded in a sheathing.

10. The pad according to claim 9, wherein the first material of the pad base element has a Shore hardness of at least 10 Shore D and at most 80 Shore D, wherein the sheathing has a Shore hardness of at least 10 Shore OO and at most 50 Shore OO.

11. (canceled)

12. A sports aid device, orthosis, or bandage comprising a pad according to claim 1.

13. The sports aid device, orthosis, or bandage according to claim 12, comprising a pad carrier, in particular a knitted or crocheted fabric, wherein the pad carrier rests against the counter-bearing surface of the pad base element of the pad.
